(19) United States
(12) Patent Application Publication
(10) Pub. No.: US 2019/0355464 A1
(43) Pub. Date: Nov. 21, 2019

(54) SYSTEM FOR USE WITH ONE OR MORE SOURCES OF PATIENT DATA

(71) Applicant: LAUREN BROWNFIELD, Houston, TX (US)

(72) Inventor: LAUREN BROWNFIELD, Houston, TX (US)

(21) Appl. No.: 16/389,186

(22) Filed: Apr. 19, 2019

Related U.S. Application Data
(60) Provisional application No. 62/674,153, filed on May 21, 2018.

(51) Int. Cl. G16H 20/40; G16H 10/60; G16H 70/20; G16H 40/20; G16H 20/10

(52) U.S. Cl. CPC G16H 20/40; G16H 10/60; G16H 20/10; G16H 40/20; G16H 70/20

(57) ABSTRACT

A patient treatment system for medical providers and patients using patient data to input surgical reservation data and receive confirmation of surgical reservation data with pre-operative instructions and pre-operative medications a category of surgery selected by a medical provider from a library of categories of surgeries; day of surgery instructions with day of surgery medications; post-operative instructions with post-operative medications to and from an administrative processor via a network and to and from a medical provider client device and patient client device to automatically confirm surgeries, medicines, pharmacies, and provide reminders to institute pre, day of, and post-operative surgery instructions.

### CROSS REFERENCE TO RELATED APPLICATION

The present application claims priority to and the benefit of co-pending U.S. Provisional Patent Application Ser. No. 62/674,153 filed on May 21, 2018, entitled “SYSTEM FOR USE WITH ONE OR MORE SOURCES OF PATIENT DATA” This reference is hereby incorporated in its entirety.

### FIELD

The present embodiment generally relates to a system for use with one or more sources of patient-affiliated data, corresponding with a group of patients who have each scheduled a surgery and may have been prescribed a pre surgery instruction and medication, a day of surgery instruction and medication, and a post-operative instruction and medication.

### BACKGROUND

A need exists for system for use with one or more sources of patient-affiliated data, corresponding with a group of patients who have each been scheduled a surgery with easy to understand reminders to prevent death during or after a surgery.

The present embodiments meet these needs.

### BRIEF DESCRIPTION OF THE DRAWINGS

The detailed description will be better understood in conjunction with the accompanying drawings as follows:

FIG. 1 depicts a diagram of equipment used in the invention.

FIGS. 2A-2D depicts rules in the administrative computer readable memory.

FIG. 3 provides details of the Library of Categories of Surgeries.

FIG. 4 shows additional content in the computer readable memory.

The present embodiments are detailed below with reference to the listed Figures.

### DETAILED DESCRIPTION OF THE EMBODIMENTS

Before explaining the present system in detail, it is to be understood that the system is not limited to the particular embodiments and that it can be practiced or carried out in various ways.

The invention relates to a patient treatment system for medical providers and patients using patient data to input surgical reservation data and receive confirmation of surgical reservation data with pre-operative instructions and pre-operative medications a category of surgery selected by a medical provider from a library of categories of surgeries; day of surgery instructions with day of surgery medications; post-operative instructions with post-operative medications to and from an administrative processor via a network and to and from a medical provider client device and patient client device to automatically confirm surgeries, medicines, pharmacies, and provide reminders to institute pre, day of, and post-operative surgery instructions.

The invention stops death by preventing patients from continuing concurrent medications that could cause complications in surgery.

Diabetic patients that are unable to be controlled with oral medication often have to use insulin to control blood glucose. Patients having surgery with sedation are unable to eat 6 hours prior to their surgery, and should not take their insulin in the morning. To take their insulin in the morning. To take their insulin in the morning and not have breakfast could lead to a significant drop in blood glucose and a diabetic coma and/or death. This invention would remind patients not to take their insulin the morning of surgery therefore prevent complications or death during surgery.

The inventions stops death by preventing people from bleeding out due to complications from surgery by stopping pre surgery the use of blood thinners.

Patients who are a stroke risk are managed with blood thinners. Physicians managing blood thinners often recommend patients to stop blood thinners prior to surgery to prevent bleeding complications during surgery and post-operative emergencies. This invention facilitates medical consults with the physicians managing medications that could lead to complications or death during or following surgery. The invention would remind the patient to stop the Apixaban 3 days prior to surgery, preventing bleeding complications during surgery and post operatively.

The invention stops suicide by preventing people from taking too many opioids or certain opioids due to allergies or other concurrent medication consumption.

The increase in opioid overdose deaths has been dramatic, and opioids were responsible for 49,000 of the 72,000 drug overdose deaths overall in the US in 2017. This invention would recommend the patient to take non-opiate alternatives as direction reducing the need for opiates for post-operative pain management. For those patients requiring opiates for pain management this invention to facilitate the prescription process with the pharmacy, reducing the prescription abuse. This invention reminds the patient of the proper dosage and regiment preventing abuse and overdose.

The invention prevents death by repeatedly and automatically generating requests for post-operative appointments so that a qualified medical professional can assess wound healing, preventing sepsis and amputations.

Patients can have post-operative complications that can be damaging and/or life-threatening. This invention will remind patients of their post-operative appointments to help prevent post-operative infections or identify post-op complications early prior to the complications and/or death.

The following terms shall be used:

The term “administrative computer readable memory” refers to data storage which is non-evanescent and configured to store sets of rules and logic for use by a processor.

The term “administrative processor” can refer to bidirectional communication device capable of receiving data and executing computer instructions, such as a lap top, computer, wearable computer, a combination of cloud based processors, a server or a similar processing device.

The term “biometric reader” refers to one or more devices adapted to determine identity of a patient by matching physical characteristics, including matching a stored personal file of a patient fingerprint, a voice print and/or a retinal image.

The term “client device” as used herein can refer to a mobile phone, smart phone, laptop, tablet, wearable computer or wristwatch with processing and memory and a display and can identify itself with a unique address and a GPS location and communicate via a network.

The term “confirmation of surgical reservation data” refers to a transmittal to the patient of the date and time and details of the surgery and the return reply by the patient agreeing to the surgery reservation data.

The term “database containing patient data” refers to a relational interactive database specifically containing pre-surgery medical condition of a patient such as the patient has diabetes; a preferred pharmacy such as Walgreens™; and surgical reservation data such date, time, year and surgical site such as Apr. 11, 2019 at 9 am at Suite 210 located at 123 Main Street Houston, Tex.

The term “day of surgery instructions” refers to a set of rules that a patient must comply with based on a specific surgery at a pre-determined time including but not limited to medications required to be taken or avoided moments before the surgery, patient behaviors modifications at the time of surgery, such as “elevate the head or compress the legs.

The term “invite” shall refer to a text message or a pdf file which is transmitted the patient client device, and may include a slide show of information on the surgery that the patient is contemplating and a form allowing the patient to request a surgery, such as dental implant placement.

The term “library of categories of surgeries” refers to a database of surgery information which includes but is not limited to pre-operative instructions such as “start antibiotics 3 days before surgery” or “stop aspirin 5 days before surgery”. The database can include lists of pre-operative medications such as ibuprofen, amoxicillin with dosage amounts and times for taking dosages given dosage amounts. The Library of categories of surgeries include; day of surgery instructions such as “do not eat within 6 hours of surgery” and day of surgery medications such as “take Augmentin twice a day after surgery; and post-operative instructions such as “don't drive the car until the following day after surgery” and “eat soft food” or “don't blow nose” with post-operative medications such as take TYLENOL3™ daily until pain dissipates.

The term “medical provider” refers to a name of a doctor, names of medical personnel working for the doctor.

The term “medical record identification number” refers to an automatically generated number when a patient desires to have surgery.

The term “medication” refers to a medicine taken by a patient with defined dosage amounts at preset times, as prescribed or supervised by a medical provider, such as Amoxicillin 4 times a day 3 days before surgery.

The term “network” refers to a local area network, a wide area network, a cellular network, a satellite network, or a global communication network.

The term “patient data” includes but is not limited to applicable pre-surgery medical condition of a patient such as the patient has type II Diabetes; a preferred pharmacy such as WALGREENS™; all concurrent medicine such as aspirin, SYNTHROID™ high blood pressure medicine, and the like not related to the surgery, and includes surgical reservation data.

The term “patient personal identification number” refers to a log in or pin number or biometric file specific to an individual patient and linked to the patient data. The term can include alpha numeric combinations, all numeric combinations, all alpha combinations, biometric files including retinal images and voice prints.

The term “patient surgery application” refers to a set of rules to manage pre-surgery, day of surgery and post-operative surgery patient procedures, medications, and patient instructions.

The term “plurality of pharmacies” refers to a database of local and national pharmacies with addresses, names, phone numbers and fax numbers for communication of patient information.

The term “post-operative instructions” refers to a set of rules that a patient must comply with based on a specific surgery at a pre-determined time including but not limited to medications required to be taken or avoided, patient behaviors modifications, such as not drinking, and can include scheduling and attending post-operative appointments and automatically transmitting reminders to attend a post-operative appointment or call the office to reschedule the appointment.

The term “preferred pharmacy” refers to a patient selected pharmacy that may or may not be in the library of pharmacies.

The term “pre-operative instructions” refers to a set of rules that a patient must comply with based on a specific surgery at a pre-determined time including but not limited to medications required to be taken or avoided before surgery, patient behaviors modifications before surgery, such as not drinking, and can include scheduling and attending pre-operative appointments and automatically transmitting reminders to attend a pre-operative appointment or call the office to reschedule the appointment.

The term “preset time intervals” refers to specific time and dates relative to a patient surgery reservation data and surgery date and time using a highly precise clock.

The term “pre-surgery medical condition of the patient” refers to a pre-existing condition of the patient, such as “limited blood circulation due to age—over 80 years”, or recovery from a surgery already performed, such as a hip surgery managed by another physician. This pre-existing condition could mean unremarkable medical history, no medical conditions or pre-existing health conditions.

The term “prewritten message” refers to a set of words transmitted to a client device to asking the patient to perform a specific behavior.

The term “processor” refers to a server, a cloud based server, or any device in communication with computer readable memory can compute according to a set of defined rules, and may include a laptop, a tablet, or series of laptops in communication with each other.

The term “surgical reservation data” includes a date, a time, a location, a parking garage location and pricing and the name of the doctor; and a patient digital address includes an email, a phone number or an iMESSAGE™ address.

The term “surgical site” can be a hospital, a medical suite, dental office, outpatient facility, or other office of a medical provider.

The term “verification window” refers to a display screen with a prewritten set of words asking the patient or medical provider to verify that the information is correct regarding medications or other instructions.

A system for automatically communicating with patients scheduling and having surgery, the system comprising: an administrative processor connected to a network and connected to a patient client device to receive and transmit information bi-directionally to a patient client device and a medical provider client device simultaneously; an administrative computer readable memory connected to the administrative processor comprising: a database containing patient data comprising: a pre-surgery medical condition of a patient; a preferred pharmacy; and surgical reservation data; a library of categories of surgeries; and wherein the computer readable memory stores logic to utilize a set of rules to: automatically generate an medical record identification number for patient data of a patient scheduling and having a surgery and linking the medical record identification number to the patient data while simultaneously transmitting an invite to download a patient surgery application to the patient client device by transmitting a plurality of prewritten messages to the patient client device including requesting a patient personal identification number, wherein the patient personal identification number provides access to surgical reservation data; automatically and simultaneously present a verification window to the patient client device enabling the patient scheduling and having a surgery to verify acceptance of the surgical reservation data for a patient selected surgical site and the preferred pharmacy in the patient data; automatically and simultaneously searching the library of categories of surgeries and generating pre-operative instructions with pre-operative medications matching the pre-surgery medical condition of a patient scheduling and having a surgery and transmitting to the medical provider client device and requesting verification from the medical provider client device that pre-operative instructions and pre-operative medication has been received and either accepted or modified, and wherein the pre-operative medications are automatically prescribed to the preferred pharmacy; automatically and simultaneously searching the library of categories of surgeries and generating a day of surgery instructions with day of surgery medications matching the pre-surgery medical condition of the patient scheduling and having a surgery and transmitting to the medical provider client device and requesting verification from the medical provider client device that day of surgery instructions and medication has been received and either accepted or modified, and wherein the day of surgery medications are automatically prescribed to the preferred pharmacy; and automatically and simultaneously searching the library of categories of surgeries and generating a post-operative instructions with post-operative medications matching a day of surgery medical condition of a patient scheduling and having a surgery and transmitting to the medical provider client device and requesting verification from the medical provider client device that post-operative instructions and post-operative medication has been received and either accepted or modified, and wherein the post-operative medications are automatically prescribed to the preferred pharmacy.

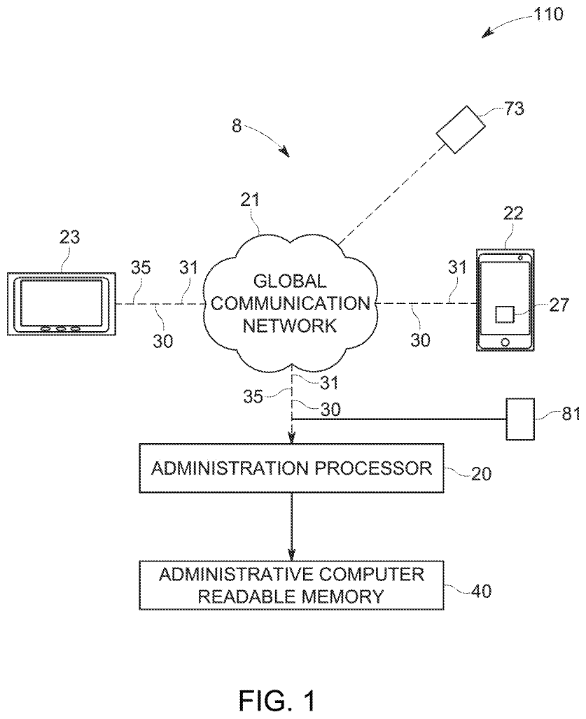

Turning now to the Figures, FIG. 1 shows an administrative processor 20 connected to a global communication network 21 further connected to a patient client device 22 and a medical provider client device 23. The administrative processor receives and transmits information bi-directionally to the patient client device, and the medical provider device simultaneously. The transmission of information can include patient data 30.

The administrative processor can also receive and transmit surgical reservation data 35 and generate confirmation of the surgical reservation data 31 to the medical provider client device and the patient client device simultaneously.

The administrative processor communicates with an administrative computer readable memory 40 connected to the administrative processor.

FIG. 1 shows a bidirectional communication link to a financial processor 73 from the administrative processor.

FIG. 1 depicts an encryption module 81 connected to the administrative processor providing bidirectional encryption and decryption of patient data as the administrative processor automatically and simultaneously generates and receives communications to and from the patient client device and medical provider client device.

FIG. 1 depicts a biometric reader 27 in one of the client devices providing verification from at least one of the client device of a user.

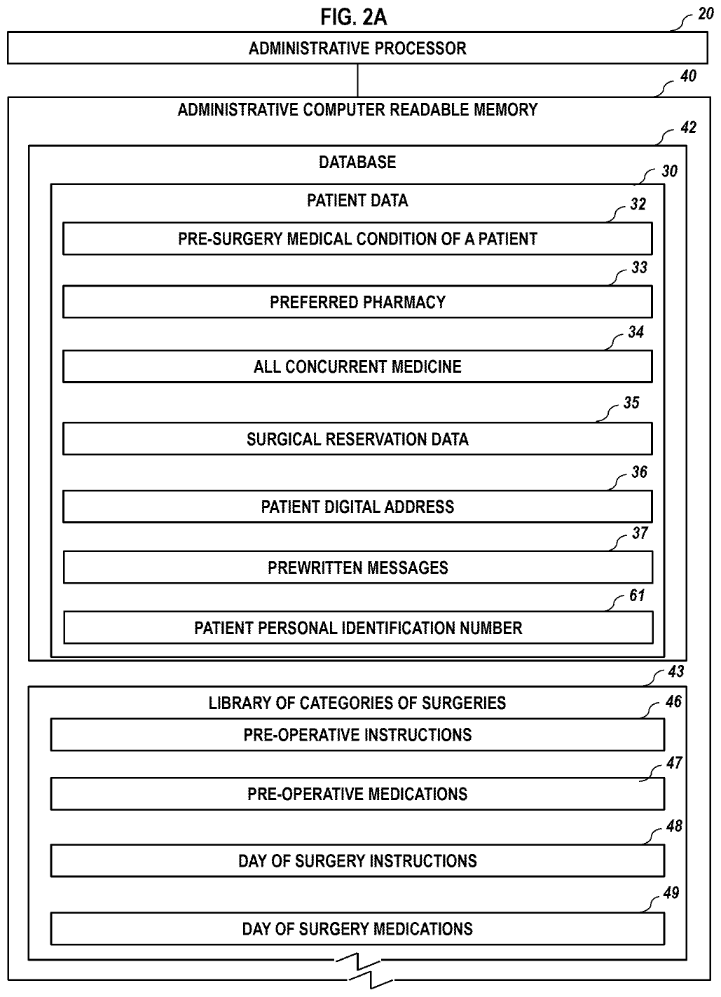

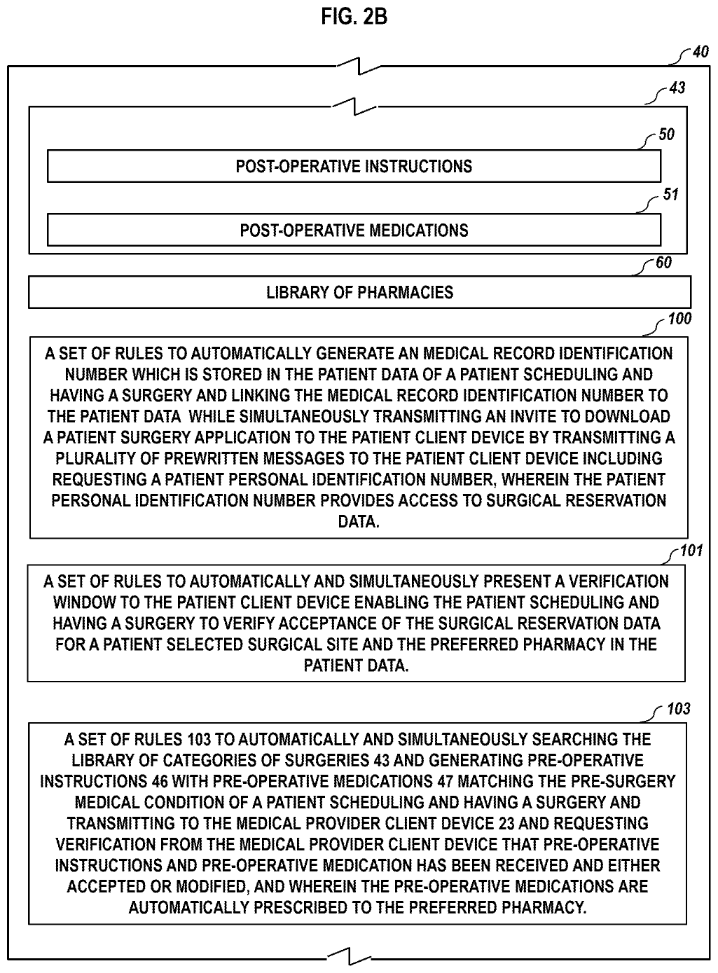

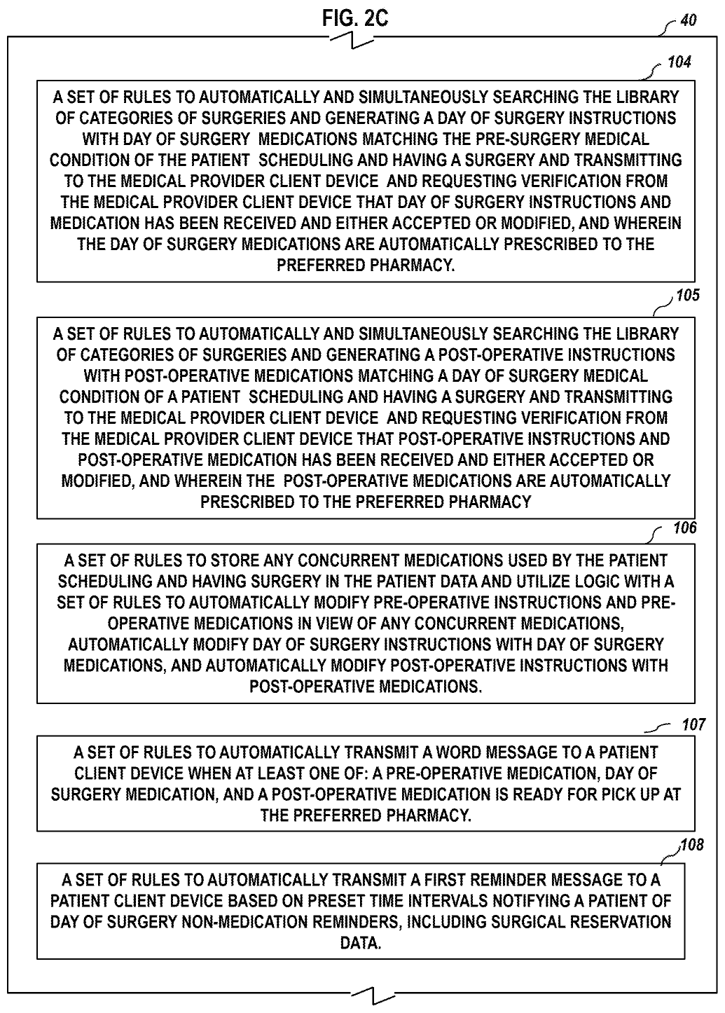

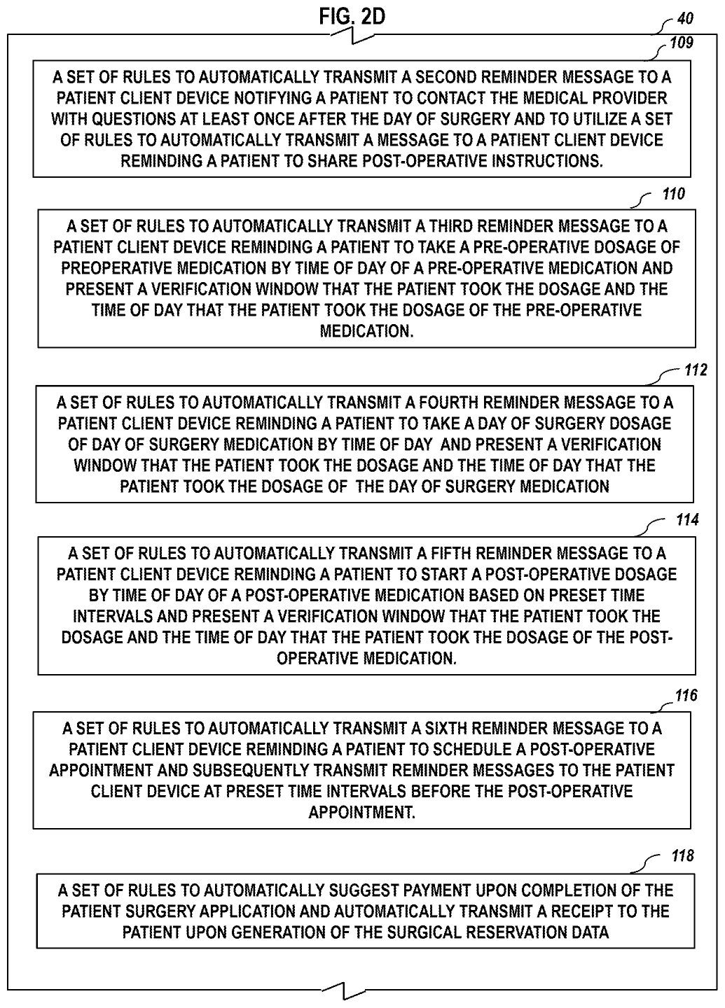

FIGS. 2A-2D depict a diagram of the administrative computer readable memory 40 according to the invention.

The administrative processor 20 is in communication with the administrative computer readable memory 40. The administrative processor and administrative computer readable memory can be a Personal computer or a MAC computer with 5 terabytes of memory.

The administrative computer readable memory 40 includes a database 42 for, containing the patient data 30. The database 42 can be a Sybase-iSQL database.

The database has prewritten messages 37, which are transmitted to a patient client device, such as “your surgical appointment is reserved for May 9, 2018” and “click here for more information.” The prewritten messages can advise a patient that surgical reservation data 35 can be viewed.

The patient data 30 contains not only the prewritten message 37 and surgical reservation data 35, but also applicable pre-surgery medical condition of a patient 32, such as congestive heart failure.

The patient data 30 includes a preferred pharmacy 33, such as a CVS™ at a certain geographic location.

The patient data 30 contains all concurrent medicine 34, such as LISINOPRIL™.

The patient data 30 includes surgical reservation data 35, such as “May 9, 2018 at 7:30 am at 6400 Fannin Street, Suite 1500, Houston, Tex., 77030 for tooth extraction”.

The patient data 30 includes a patient digital address 36, such as a text address of 7137 Candy Lane or an email address such as BettyBoop@gmail.com.

The patient data 30 contains a patient personal identification number 61.

The administrative computer readable memory 40 can include the library of categories of surgeries 43.

The Library of Categories of Surgeries 43 includes pre-operative instructions 46, such as start an antibiotic 24 hours before surgery.

The library of categories of surgeries 43 includes pre-operative medications 47, such as the antibiotic known as CLARITHROMYCIN™.

The library of categories of surgeries 43 includes day of surgery instructions 48, such as no eating 6 hours before surgery.

The library of categories of surgeries 43 includes day of surgery medications 49, such as TYLENOL 3™ start 4 to 6 hours after surgery.

The library of categories of surgeries 43 includes post-operative instructions 50, such as start a steroid 24 hours after surgery, no nose blowing, no sucking straws, and eat soft foods.

The library of categories of surgeries 43 includes with post-operative medications 51, such as a MEDROL DOSE PAK™ 5 mg starting 12 hours after surgery.

A library of pharmacies 60 is in the administrative computer readable memory 40 and includes names of pharmacies, Tschiffely pharmacy, addresses, such as 1145 19th Street, NW Washington, D.C., 20036, hours of operation, such as 8:30-6 pm; dates closed, such as Saturday and Sunday, phone numbers such as 202-466-8440, directions, website, ratings, and reviews.

In the administrative computer readable memory 40 are a plurality of rules, namely, rules to do several different steps.

The administrative computer readable memory contains logic to implement a set of rules 100 to automatically generate an medical record identification number 29 which is stored in the patient data 30 of a patient scheduling and having a surgery and linking the medical record identification number 29 to the patient data 30 while simultaneously transmitting an invite 97 to download a patient surgery application 99 to the patient client device 22 by transmitting a plurality of prewritten messages 37 to the patient client device including requesting a patient personal identification number 61, wherein the patient personal identification number provides access to surgical reservation data.

The administrative computer readable memory contains logic to implement a set of rules 101 to automatically and simultaneously present a verification window 72 to the patient client device 22 enabling the patient scheduling and having a surgery to verify acceptance of the surgical reservation data for a patient selected surgical site 28 and the preferred pharmacy 33 in the patient data.

The administrative computer readable memory contains logic to implement a set of rules 103 to automatically and simultaneously searching the library of categories of surgeries 43 and generating pre-operative instructions 46 with pre-operative medications 47 matching the pre-surgery medical condition of a patient scheduling and having a surgery and transmitting to the medical provider client device 23 and requesting verification from the medical provider client device that pre-operative instructions and pre-operative medication has been received and either accepted or modified, and wherein the pre-operative medications are automatically prescribed to the preferred pharmacy.

The administrative computer readable memory contains logic to implement a set of rules 104 to automatically and simultaneously searching the library of categories of surgeries and generating a day of surgery instructions 48 with day of surgery medications 49 matching the pre-surgery medical condition of the patient scheduling and having a surgery and transmitting to the medical provider client device 23 and requesting verification from the medical provider client device that day of surgery instructions and medication has been received and either accepted or modified, and wherein the day of surgery medications are automatically prescribed to the preferred pharmacy.

The administrative computer readable memory contains logic to implement a set of rules 105 to automatically and simultaneously searching the library of categories of surgeries and generating a post-operative instructions 50 with post-operative medications 51 matching a day of surgery medical condition of a patient scheduling and having a surgery and transmitting to the medical provider client device 23 and requesting verification from the medical provider client device that post-operative instructions and post-operative medication has been received and either accepted or modified, and wherein the post-operative medications are automatically prescribed to the preferred pharmacy.

The administrative computer readable memory contains logic to implement a set of rules 106 a set of rules to store any concurrent medications 34 used by the patient scheduling and having surgery in the patient data 30 and utilize logic with a set of rules 53 to automatically modify pre-operative instructions and pre-operative medications in view of any concurrent medications, automatically modify day of surgery instructions with day of surgery medications, and automatically modify post-operative instructions with post-operative medications.

The administrative computer readable memory contains logic to implement a set of rules to automatically transmit a word message 107 to a patient client device when at least one of: a pre-operative medication, day of surgery medication, and a post-operative medication is ready for pick up at the preferred pharmacy.

The administrative computer readable memory contains logic to implement a set of rules to automatically transmit a first reminder message 108 to a patient client device based on preset time intervals notifying a patient of day of surgery non-medication reminders, including surgical reservation data.

The administrative computer readable memory contains logic to implement a set of rules to automatically transmit a second reminder message 109 to a patient client device notifying a patient to contact the medical provider with questions at least once after the day of surgery and to utilize a set of rules to automatically transmit a message to a patient client device reminding a patient to share post-operative instructions.

The administrative computer readable memory contains logic to implement a set of rules to automatically transmit a third reminder message 110 to a patient client device reminding a patient to take a pre-operative dosage of preoperative medication 57 by time of day of a pre-operative medication and present a verification window that the patient took the dosage and the time of day that the patient took the dosage of the pre-operative medication.

The administrative computer readable memory contains logic to implement a set of rules to automatically transmit a fourth reminder message 112 to a patient client device reminding a patient to take a day of surgery dosage of day of surgery medication by time of day and present a verification window that the patient took the dosage and the time of day that the patient took the dosage of the day of surgery medication

The administrative computer readable memory contains logic to implement a set of rules to automatically transmit a fifth reminder message 114 to a patient client device reminding a patient to start a post-operative dosage by time of day of a post-operative medication based on preset time intervals and present a verification window that the patient took the dosage and the time of day that the patient took the dosage of the post-operative medication.

The administrative computer readable memory contains logic to implement a set of rules to automatically transmit a sixth reminder message 116 to a patient client device reminding a patient to schedule a post-operative appointment and subsequently transmit reminder messages to the patient client device at preset time intervals before the post-operative appointment.

FIG. 2A-D depicts the administrative computer readable memory containing logic to implement a set a set of rules 118 to automatically suggest payment 74 upon completion of the patient surgery application and automatically transmit a receipt to the patient upon generation of the surgical reservation data

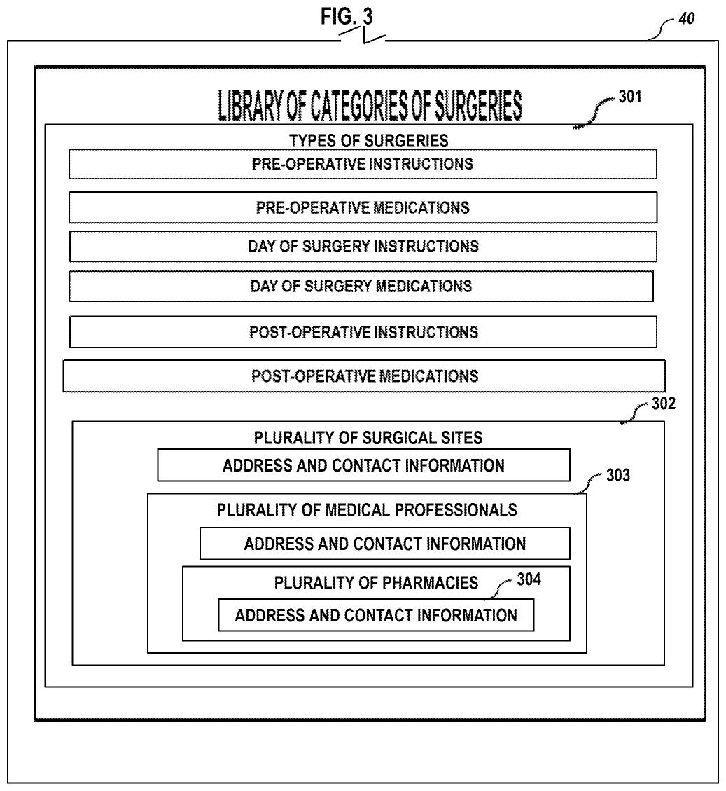

FIG. 3 is a detail of the Library of Categories of Surgeries containing in the computer readable memory 40 a plurality of types of surgeries 301, wherein each type of surgery has pre-operative instructions with pre-operative medications; day of surgery instructions with day of surgery medications; post-operative instructions with post-operative medications; a plurality of surgical sites 302, each site having and address and contact information; a plurality of medical professionals 303 by type of surgery, each medical professional having an address and contact information; and a plurality of pharmacies 304, each pharmacy having an address and contact information.

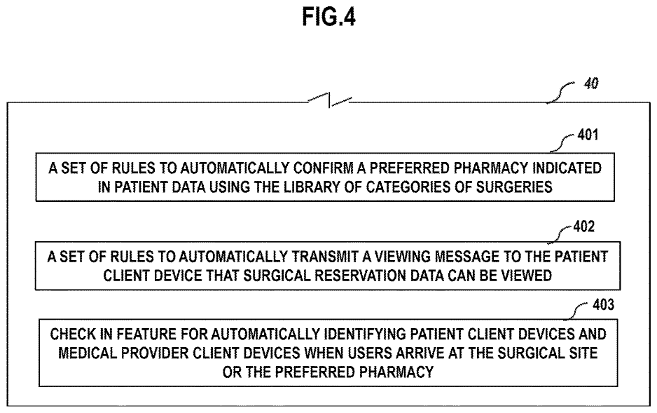

FIG. 4 shows additional content in the computer readable memory 40.

The administrative computer readable memory contains logic to implement a set of rules to automatically confirm a preferred pharmacy 401 indicated in patient data using the library of categories of surgeries, and a set of rules to automatically transmit a viewing message 402 to the patient client device that surgical reservation data can be viewed.

Embodiments include a dynamic check in feature 403 for automatically identifying patient client devices and medical provider client devices when users arrive at the surgical site or the preferred pharmacy.

In embodiments of the system, the dynamic check in feature provides the automatic messages in real time to the administrative processor and the plurality of medical provider client devices for automatically tracking of arrivals of pluralities of patient client devices at pluralities of surgical sites and pluralities of preferred pharmacies simultaneously.

### Example One

A system for use by an oral surgery office for a group of patients having oral surgery.

The surgeon's office connects an administrative processor connected to a global communication network like the Internet.

Patients can connect to the system using a smart phone as their patient client device to receive and transmit information bi-directionally to the surgeon's office and the administrative processor simultaneously.

The patient provides patient data to a medical provider client device at the doctor's office, such as Dr. Brownfield.

For example, for Mary, age 32 needs wisdom teeth removed. Mary would input her applicable pre-surgery medical condition of the patient, such as she had a miscarriage 3 months prior to the surgery, her weight, 130 pounds, and other details on medical history. Mary inputs her patient digital address, such as an email. She inputs that her preferred pharmacy is WALGREENS™ at the corner of Montrose and Hawthorne. Mary inputs that her concurrent medicine is birth control and SYNTHROID™.

After being viewed by the medical provider and deciding on a surgery, the medical provider inputs to Mary's patient data, surgical reservation data including the type of surgery, the day of surgery, the time of surgery, the location of the surgery, the length of time expected for the surgery and other details.

In the administrative computer readable memory is a database containing the patient data and a library of categories of surgeries including the category of surgery for wisdom teeth removal.

The system for Mary uses logic and a set of rules to: automatically generate a patient identification number for Mary, such as MaryC1345, and then link MaryC1345 to her patient data.

The system for Mary uses logic and a set of rules to automatically confirm a preferred pharmacy, the Walgreens at Montrose and Hawthorne using Mary's patient data and the library of pharmacies.

The system for Mary uses logic and a set of rules to automatically transmit pre-operative instructions with pre-operative medications to her Dr. Brownfield's medical provider client device using a category of surgery selected by the medical provider from the library of categories of surgeries.

The system for Mary uses logic and a set of rules to automatically present to Dr. Brownfield a verification window on her office laptop verifying acceptance of the pre-operative instructions with pre-operative medications and prompting the medical provider to customize the pre-operative instructions with pre-operative medications prior to actuating any subsequent steps.

The system for Mary uses logic and a set of rules to automatically transmit prior to and on the day of surgery very specific day of surgery instructions with day of surgery medications to Dr. Brownfield for the doctor to confirm as correct or to modify given patient data.

The system for Mary uses logic and a set of rules to automatically present a verification window to Dr. Brownfield's office computer to verify acceptance of the day of surgery instructions with day of surgery medications and prompt the medical provider to customize the day of surgery instructions with day of surgery medications prior to actuating any subsequent steps.

The system for Mary uses logic and a set of rules to automatically transmit post-operative instructions with post-operative medications to Dr. Brownfield and automatically present a verification window to the medical provider client device verifying acceptance of the post-operative instructions with post-operative medications and prompting the medical provider to customize the post-operative instructions with post-operative medications prior to actuating any subsequent steps.

The system for Mary uses logic and a set of rules to automatically use the patient identification number such as: MaryC1345 to transmit a message to download a patient surgery application from the administrative computer readable memory to the patient client device by transmitting one of a plurality of prewritten messages to the patient client device requesting a personal identification number.

The patient surgery application is resident in administrative computer readable memory connected to the administrative processor. Portions of the patient surgery application can be downloaded to Mary's cell phone.

Mary transmits a personal identification number BettyBoop1 to the administrative processor enabling Mary to access surgical reservation data on her cell phone.

The system for Mary uses logic and a set of rules to automatically link the personal identification number BettyBoop1 to the patient data of Mary.

The system for Mary uses logic and a set of rules to automatically transmit a preset message to Mary's cell phone that surgical reservation data can be viewed.

It should be noted that the system automatically presents a verification window to Mary's cell phone enabling a patient to verify acceptance of the surgical reservation data and preferred pharmacy.

Automatically, after Mary confirms her surgery reservation and her preferred pharmacy, the administrative processor will use logic to operate on a set of rules to provide very specific wisdom teeth removal pre-operative instructions with wisdom teeth removal pre-operative medications. Mary is told to go to the preferred pharmacy and obtain amoxicillin which has been automatically transmitted to the preferred pharmacy by the administrative processing using patient data.

Before the surgery, Mary will receive on her patient client device day of surgery instructions with day of surgery medications.

The message would arrive at least 24 hours before the surgery. This message could include the address (such as 90 Main Street Houston, Tex.), the time, such as 2 pm, and the geographic collection point, such as East Entrance, to collect the patient after surgery, and the option to share this information with Mary's responsible party.

Additionally after the surgery or day of surgery Mary will get post-operative instructions with post-operative medications. The post-operative instructions will include interactive acknowledgement of the instructions on a step by step basis to ensure safety of the patient and quick recovery with minimal complications, as well as the option to share post-operative instructions and post-operative medications with Mary's responsible party.

The system uses logic to utilize a set of rules to automatically transmit a message to Mary's patient client device reminding Mary to start post-operative medications based on preset time intervals, such as “Take first dose now” and the time is 12 hours following surgery.

Not only does the system remind Mary to take a dosage by time of day, but the system presents a verification window to Mary's cell phone enabling Mary to verify that she took her specific dosage of post-operative medication on a specific day and at a specific time.

The system reminds Mary to repeat the last two steps of reminding and verifying the taking of the post-operative dosage amounts until the post-operative medication is no longer needed.

### Example 2

A system for automatically communicating with patients scheduling and having surgery using 4 cell phones.

An administrative processor that is a Dell PC is in communication with the cellular network, and the Internet for email. The administrative processor can be accessed by each of the 4 cell phones, namely an IPhone™ for a patient Bob, trying to schedule surgery as his patient client device, an Orthopedic surgeon, Dr. White has the second cell phone, an anesthesiologist has the third cell phone, and the wife of the patient has the 4^{th} cell phone. All cell phones and the administrative processor can receive and transmit information bi-directionally to a patient client device and to all the medical provider client devices simultaneously.

The patient is interested in bunion surgery as he has severe pain in his right foot.

The patient communicates using a digital address to the administrative processor which has connected administrative computer readable memory of 512 Gig of memory.

In the memory is a database, a dynamic interactive HIPPA compliant Oracle™ database containing patient data for Bob including his pre-surgery medical condition of short term and long term memory loss; a preferred pharmacy which is GreenTree™ pharmacy and surgical reservation data for Jun. 14, 2019 at 7 am. The memory contains a library of categories of surgeries including “foot surgery” as a category which contains “bunions”.

The computer readable memory stores logic to utilize a set of rules to: automatically generate an medical record identification number P-100 for patient data of Bob.

The set of stored rules links Bob's medical record identification number to his patient data in the database while simultaneously transmitting an invite to Bob's client device instructing Bob to download a patient surgery application. He receives this instruction in a group of prewritten messages namely “Bob, please download the patient surgery application by clicking on the link below”.

Additionally, he receives an instruction on his cell phone to create a patient personal identification number a log in number such as 44Graham$., wherein the patient personal identification number provides access to surgical reservation data stored in the memory.

A set of stored rules in the memory automatically and simultaneously present a verification window of surgical reservation data displaying date, time, and type of surgery, location of surgery with an address, and names of the surgeon to the patient client device, that is, Bob's cellular phone. This verification window enables Bob to verify acceptance of the surgical reservation data for a patient selected surgical site and the preferred pharmacy in the patient data by pushing a displayed button labelled “confirm”, or a clickable phone number to call the office of the surgeon.

If Bob, verifies the surgical reservation data automatically and simultaneously the administrative processor searches the library of categories of surgeries and generates pre-operative instructions for Bob with pre-operative medications that Bob needs to take that match Bob's pre-surgery medical condition. Bob got a preoperative message of no eating or drinking within 6 hours of surgery, no preoperative medications needed, a driver to take Bob home is required.

Additionally, the medical provider client device receives the verification of the surgical reservation data and verifies the pre-operative instructions and the lack of pre-operative medication.

The administrative processor automatically and simultaneously searches the library of categories of surgeries and generates a day of surgery instructions for Bob with day of surgery medications including matching the pre-surgery medical condition of Bob. Specifically Bob, with memory issues, is given no day of surgery medication. Bob is automatically given another reminder not to wear makeup, wear loose clothing on day of surgery, and do not eat or drink until after surgery and a driver must be present.

Verification is requested from the surgeon confirming the day of surgery instructions and day of surgery medication. The surgeon however, decides to modify the day of surgery instructions to read that Bob must share these instructions with the wife, that is, the fourth cell phone holder. The modified instructions are transmitted from the surgeon to the administrative processor and then to Bob, asking Bob to “share” the day of surgery instructions with his wife.

On the day of surgery, automatically and simultaneously the administrative processor uses rules to search the library of categories of surgeries and generating a post-operative instruction with post-operative medication to both Bob and Bob's wife based on Bob sharing the digital address of his wife's phone back to the database and his patient data in the database.

Bob is going to be prescribed Tramadol™ for post-operative pain management using the rules of the administrative processor.

The proposed prescription is transmitting to the medical provider client device and verification from the medical provider client device is requested that post-operative instructions and post-operative medication is accepted, and wherein the post-operative medications are automatically prescribed to the preferred pharmacy for Bob or Bob's wife to pick up.

In Bob's situation, reminders to take the medication are transmitted at preset intervals to his cell phone, so he does not overdose.

For Bob, the medical provider has modified the instructions to require automatic notice for Bob to take the pill and present a button that confirms he took the pills, which records the date and time of ingestion.

Additional reminders are modified in time, based on the moment when Bob presses the button confirming when he took the post-operative pill.

While these embodiments have been described with emphasis on the embodiments, it should be understood that within the scope of the appended claims, the embodiments might be practiced other than as specifically described herein.

1. A system for automatically communicating with patients scheduling and having surgery, the system comprising:
a. an administrative processor connected to a network and connected to a patient client device to receive and transmit information bi-directionally to a patient client device and a medical provider client device simultaneously;
b. an administrative computer readable memory connected to the administrative processor comprising:
1. a database containing patient data comprising: a pre-surgery medical condition of a patient; a preferred pharmacy; and surgical reservation data;
2. a library of categories of surgeries; and
wherein the computer readable memory stores logic to utilize a set of rules to:
i. automatically generate an medical record identification number for patient data of a patient scheduling and having a surgery and linking the medical record identification number to the patient data while simultaneously transmitting an invite to download a patient surgery application to the patient client device by transmitting a plurality of prewritten messages to the patient client device including requesting a patient personal identification number, wherein the patient personal identification number provides access to surgical reservation data;
ii. automatically and simultaneously present a verification window to the patient client device enabling the patient scheduling and having a surgery to verify acceptance of the surgical reservation data for a patient selected surgical site and the preferred pharmacy in the patient data;
iii. automatically and simultaneously searching the library of categories of surgeries and generating pre-operative instructions with pre-operative medications matching the pre-surgery medical condition of a patient scheduling and having a surgery and transmitting to the medical provider client device and requesting verification from the medical provider client device that pre-operative instructions and pre-operative medication has been received and either accepted or modified, and wherein the pre-operative medications are automatically prescribed to the preferred pharmacy;
iv. automatically and simultaneously searching the library of categories of surgeries and generating a day of surgery instructions with day of surgery medications matching the pre-surgery medical condition of the patient scheduling and having a surgery and transmitting to the medical provider client device and requesting verification from the medical provider client device that day of surgery instructions and medication has been received and either accepted or modified, and wherein the day of surgery medications are automatically prescribed to the preferred pharmacy; and
v. automatically and simultaneously searching the library of categories of surgeries and generating a post-operative instructions with post-operative medications matching a day of surgery medical condition of a patient scheduling and having a surgery and transmitting to the medical provider client device and requesting verification from the medical provider client device that post-operative instructions and post-operative medication has been received and either accepted or modified, and wherein the post-operative medications are automatically prescribed to the preferred pharmacy.

2. The system of claim 1, wherein the computer readable memory stores logic to utilize a set of rules to store concurrent medications used by the patient scheduling and having surgery in the patient data and utilize logic with a set of rules to automatically modify pre-operative instructions and pre-operative medications in view of any concurrent medications, automatically modify day of surgery instructions with day of surgery medications, and automatically modify post-operative instructions with post-operative medications.

3. The system of claim 1, wherein the computer readable memory stores logic to utilize a set of rules to automatically transmit a word message to a patient client device when at least one of: a pre-operative medication, day of surgery medication, and a post-operative medication is ready for pick up at the preferred pharmacy.

4. The system of claim 1, wherein the computer readable memory stores logic to utilize a set of rules to automatically transmit a first reminder message to a patient client device based on preset time intervals notifying a patient of day of surgery non-medication reminders, including surgical reservation data.

5. The system of claim 1, wherein the patient data includes a patient digital address.

6. The system of claim 1, wherein the computer readable memory stores logic to utilize a set of rules to automatically transmit a second reminder message to a patient client device notifying a patient to contact the medical provider with questions at least once after the day of surgery and to utilize a set of rules to automatically transmit a message to a patient client device reminding a patient to share post-operative instructions.

7. The system of claim 1, wherein the computer readable memory stores logic to utilize a set of rules to automatically transmit a third reminder message to a patient client device reminding a patient to take a pre-operative dosage of pre-operative medication by time of day of a pre-operative medication and present a verification window that the patient took the dosage and the time of day that the patient took the dosage of the pre-operative medication.

8. The system of claim 1, wherein the computer readable memory stores logic to utilize a set of rules to automatically transmit a fourth reminder message to a patient client device reminding a patient to take a day of surgery dosage of day of surgery medication by time of day and present a verification window that the patient took the dosage and the time of day that the patient took the dosage of the day of surgery medication

9. The system of claim 1, wherein the computer readable memory stores logic to utilize a set of rules to automatically transmit a fifth reminder message to a patient client device reminding a patient to start a post-operative dosage by time of day of a post-operative medication based on preset time intervals and present a verification window that the patient took the dosage and the time of day that the patient took the dosage of the post-operative medication.

10. The system of claim 1, wherein the computer readable memory stores logic to utilize a set of rules to automatically transmit a sixth reminder message to a patient client device reminding a patient to schedule a post-operative appointment and subsequently transmit reminder messages to the patient client device at preset time intervals before the post-operative appointment.

11. The system of claim 1, wherein the library of categories of surgeries comprises:
a. a plurality of types of surgeries, wherein each type of surgery has pre-operative instructions with pre-operative medications; day of surgery instructions with day of surgery medications; post-operative instructions with post-operative medications;
b. a plurality of surgical sites, each site having and address and contact information;
c. a plurality of medical professionals by type of surgery, each medical professional having an address and contact information; and
d. a plurality of pharmacies, each pharmacy having an address and contact information.

12. The system of claim 1, wherein the computer readable memory stores logic to utilize a set of rules to automatically confirm a preferred pharmacy indicated in patient data using the library of categories of surgeries.

13. The system of claim 1, wherein the computer readable memory stores logic to utilize a set of rules to automatically transmit a viewing message to the patient client device that surgical reservation data can be viewed.

14. The system of claim 1, comprising a bidirectional communication link to a financial processor from the administrative processor, and wherein the computer readable memory stores logic to utilize a set of rules to automatically suggest payment upon completion of the patient surgery application and automatically transmit a receipt to the patient upon generation of the surgical reservation data.

15. The system of claim 1, comprising an encryption module connected to the administrative processor providing bidirectional encryption and decryption of patient data as the administrative processor automatically and simultaneously generates and receives communications to and from the patient client device and medical provider client device.

16. The system of claim 1, comprising a dynamic check in feature for automatically identifying patient client devices and medical provider client devices when users arrive at the surgical site or the preferred pharmacy.

17. The system of claim 16, wherein the dynamic check in feature provides the automatic messages in real time to the administrative processor and the plurality of medical provider client devices for automatically tracking of arrivals of pluralities of patient client devices at pluralities of surgical sites and pluralities of preferred pharmacies simultaneously.

18. The system of claim 1, wherein the verification from at least one of the client device uses a biometric reader.

19. The system of claim 1, wherein the preferred pharmacy is selected from a Library of Pharmacies in the administrative computer readable memory.
